(19) United States
(12) Patent Application Publication
(10) Pub. No.: US 2006/0106004 A1
(43) Pub. Date: May 18, 2006

(54) Unique methods and formulations of bio-identical sex steroids for the treatment of pathophysiologic aberrations of menopause

(76) Inventors: Steven A. Brody, San Diego, CA (US); Jerry Dodd, Escondido, CA (US)

Correspondence Address: Steven A. Brody, Suite 108, 6719 Alvarado Rd., San Diego, CA, 92037, US

(21) Appl. No.: 10/987,782

(22) Filed: Nov. 12, 2004

(51) Int. Cl. A61K 31/56

(52) U.S. Cl. 514/170; 514/182

(57) ABSTRACT

Consistent relief of physical and psychological disturbances in menopause is achieved by the administration of estrogenic hormones, which are bio-identical to natural estradiol and estriol, in a ratio ranging from 1.1:1.0 to 10.0:1.0, either with or without the combined administration of testosterone in a ratio of total estrogen to testosterone ranging from 11.0:1.0 to 0.9:1.0. These combinations of sex steroids may be administered either on a continuous basis or on a cyclic basis consisting of 21-30 days out of the month for physiological adaptation.

### BACKGROUND OF THE INVENTION

This invention relates to unique methods and formulations of bio-identical sex steroids for the treatment of pathophysiologic aberrations of menopause.

Estrogens have several important health benefits. The amelioration of estrogen-deficiency symptoms in women, including hot flashes, sleeplessness, fatigue and sexual dysfunction, are often dramatic with estrogen. Estrogen retards and may prevent the progression of menopausal osteoporosis, thus preventing hip fractures and compression of spinal vertebrae. Some estrogens may, in fact, retard the onslaught of memory disturbances in the older age population. Outbreaks of perspiration, dizziness, and palpitations can all be ameliorated with the use of estrogen therapy.

Psychological changes in women are often seen around the time of the menopause. These changes may manifest themselves as mood disturbances, emotional imbalances or sensitivities, anxiety, depression and stress, including a sense of hopeless, helplessness or worthlessness.

Traditional hormone replacement therapy utilizing conjugated equine estrogens, with or without synthetic progestins, has been subject to increasing clinical research scrutiny in the past five years. The landmark Women's Health Initiative (WHI) study of 17,000 women on these preparations was suspended in July of 2002 because of serious side effects. The WHI study showed that these drugs had little or no benefit to the heart, brain or quality of life scores of postmenopausal women. On the contrary, the women in this study had a significantly increased risk of breast cancer, ovarian cancer, dementia, asthma and cardiovascular problems, including strokes and heart attacks. In March of 2004 the FDA halted a seven-year study of 11,000 women in the estrogen-only arm of the WHI research protocol because of an increased risk of stroke. Many of the findings of the WHI study were unanticipated or unexpected because of previous scientific findings suggesting a beneficial effect of estrogen on lipid profiles, heart disease and mentation.

The results of the Women's Health Initiative study raise the question of whether there are alternatives to traditional treatment which might offer all of the advantages of estrogen replacement therapy without major metabolic or oncogenic risks. An important treatment

option is the use of bio-identical hormone replacement therapy. Bio-identical hormones are derived from plant sources such as soy and yams, and these compounds have the same chemical structures as the sex steroids found in humans. Bio-identical sex steroids include estrogen, progesterone, testosterone and dehydroepiandrosterone (DHEA).

There are three forms of estrogen which are produced in-vivo; these are estrone (E₁), estradiol (E₂) and estriol (E₃). The potencies vary. Estradiol is the most potent estrogenic sex steroid and estriol is the least potent. These estrogens have the capacity to relieve menopausal symptoms, decrease the risk of colorectal cancer and increase bone density, leading to fewer osteoporotic fractures.

Use of the most potent estrogen, estradiol, has the advantage of generating the highest clinical efficacy and clinical responsiveness in the amelioration of hypoestrogenic symptoms. Conversely, the use of estrone has a particular disadvantage, in that experimental evidence indicates that it may have a strong correlation with the subsequent development of breast neoplasia. Estriol is the most omnipresent female estrogenic sex steroid in vivo, representing about 90% of the estrogenic composition in the female body. The administration of estradiol has the advantage of providing a natural precursor for the body's own metabolism of estradiol as a prohormone. Estradiol is, in fact, metabolized into the other estrogenic sex steroids, so that the patient can achieve both clinical efficacy and a natural distribution of sex steroids as the prohormone is metabolized.

The use of bio-identical estrogenic steroids offers the benefits of estrogen with very little or no increased risk of heart disease, cancer and stroke. When taken topically or transdermally these estrogens stabilize the inflammatory markers in blood which are associated with heart disease. They can also improve blood lipid levels

.Estriol has the distinct advantage over other estrogens, in that it may prevent or suppress the development of mastocarcinomas. Estriol has been shown to decrease the risk of breast cancer in several published medical studies Estriol has anti-mammary cancerogenic activity both in the induction and promotion phases of tumor development. Estriol has been safely given to women with metastatic breast cancer in doses from 2.5 to 15 mg/day. Over ⅓ of these women had arrested growth and remission of their metastatic lesions. Estriol is capable of reducing mammary carcinoma rates by 80% following radiation or carcinogenesis. Thus, E₃ may exert antiestrogenic benefits in normal tissue. Estriol also has a favorable effect on the lower genital tract, including the cervix, uterus, vagina and vulva. Genitally applied estriol may correct genitourinary atrophy and dyspareunia.

Limiting an estrogen regimen to estriol, or proposing a multi-estrogen therapy in which estriol is the principal component, has a definite disadvantage in that typical complaints of constitutional or psychological aberrations of menopause are not satisfactorily diminished. Additionally, Estriol may be less effective for the prophylaxis of bone demineralization in osteoporosis.

Pharmacologically, estriol is regarded as a weak-acting estrogen, although it is normally secreted during a woman's menstrual cycle. Estriol does not significantly stimulate uterine weights when administered to ovariectomized rats. It has little or no effect on the uterus in studies involving adult females. Relatively high dosages of estriol may fail to

suppress ovulation in women, even when this is clearly possible with comparable doses of estradiol. Whereas, estriol is in itself estrogenic, it may have relatively antiestrogenic effects via the displacement of more powerful estrogens from receptors on biologic targets where estrogen could pose clinical risks.

Estriol has anti-mammary carcinogenic activity both in the induction and promoter phase of tumor development. Estriol can however clear psycho-vegitative symptoms of menopause as well as local dystrophic findings in the genital tract. Genitally applied estriol may correct genital urinary atrophy and dyspareunia.

Traditional hormone replacement therapeutic regimens utilize oral administration of conjugated estrogens derived from horses, alone or in combination with synthetic progestin. Unapposed estradiol stimulates endometrial proliferation, resulting in an increased risk of endometrial cancer. Estradiol stimulation of the endometrium is usually offset by including progestin in part of the monthly cycle of therapy. Progestins are compounds, other than natural progesterone, that are able to sustain the human secretory endometrium. Unfortunately, the administration of conjugated estrogens along with progestins causes undesirable side effects including alterations of blood lipids and pro-inflammatory markers that are associated with an increased risk of coronary artery disease. A major concern is the fact that these compounds cause significant increases in the rates of breast and ovarian cancer, strokes, heart attacks and venous thromboembolism

A proportion of perimenopausal and postmenopausal women will fail to achieve adequate improvement of sexual dysfunction in response to estrogenic therapy. In these women, the anabolic steroid testosterone may be utilized. Bio-identical testosterone may be administered orally or transdermally. Testosterone may improve mood disturbances and dysphoria, may decrease hypoestrogenic symptoms, may enhance sexual responsiveness, and may have other anabolic effects. Testosterone may be safely utilized in transdermal gel preparations of estrogenic sex steroids.

It is accordingly the object of this invention to provide a method and composition for the relief from menopausal symptoms while limiting a woman's exposure to the adverse effects of estradiol therapy. This and other objects of the invention will become apparent to those of ordinary skill in the art from the following description.

### SUMMARY of INVENTION

This invention features a method of hormone replacement therapy, whereby a combination of native estradiol, native estriol, and optionally testosterone, are administered transdermally, or by a comparably effective route, to a patient in need of such therapy. The native hormones are synthesized from compounds obtained from a plant source, preferably those of the soybean family or yam plant. The substances are micronized, and dissolved in a physiologically acceptable cream or gel that can be applied to the skin or contained in an oral dosage form. Preferably the cream is applied to areas of the skin that overlie fatty tissue. Examples of such areas are the lower abdomen, inner thighs, and arms. Persons to benefit from the method of the invention are

women who are approaching, or have already come to the end of their child-bearing years. However, hormone deficiencies caused by events other than menopause can also be treated by the invention

The preferred ratio by weight, of estradiol to estriol, is approximately 1.05:1.00 to 9.0:1.0, depending on clinical requirements. Using the method and formulation described in this invention, estrogen dosing can be customized for the individual patient. The preferred concentration of total estrogens to testosterone range from 11.0:1.0. to 0.9:1.0 The addition of testosterone to the therapy is optional, based on the judgment of the physician and the desired clinical outcome. Preferably, the concentration of components in the gel is 280 mg estradiol, 120 mg estriol, 80 mg testosterone, in a total of 100 gm of gel. The daily dose will consist of a single application of 1.0 gm of formulated gel (2.8 mg estradiol, 1.2 mg estriol, with or without 0.8 mg testosterone) spread over approximately 750 cm² of skin. To formulate the therapy, micronized estradiol, estriol, and testosterone powders are measured in the above amounts; solubilized in alchohol; and mixed with enough gel to reach a final weight of 100 gms. The gel is then inserted into a pump dispenser capable of metering the prescribed daily dose for the duration of the 21-30 day treatment cycle.

Clinical observations by Steven A. Brody, M.D., Ph.D. support the conclusion that the proposed method of hormone replacement therapy (HRT) provides superior relief from menopausal symptoms than combination HRT regimens in which the estriol concentration exceeds the estradiol concentration. Furthermore, there are lower risks of adverse events than with the HRT regimen consisting of conjugated equine estrogens with a synthetic progestin. Estriol may exert anti-estrogenic effects in tissues such as the ductal epithelium of the breast, thus reducing the risks of adverse effects of E.R.T. Estrogen exerts a bone sparing effect and provides relief from hot flashes.

The described formulation, particularly when administered transdermally, has a favorable effect on blood lipids, pro-inflammatory factors, and substrates in the coagulation cascade. Thus, risk factors for coronary artery disease, vascular occlusive disease, thromboembolism and stroke are reduced. Cognitive function and relief of urogenital dystrophic symptoms may be anticipated. The addition of estriol (E₃) allows for the administration of lower doses of estradiol (E₂) while still achieving satisfactory clinical results. These aforementioned benefits are a direct effect of competitive and selective binding by estriol and estradiol to physiologically relevant estrogen receptors.

Clinical observations confirm that micronized bioequivalent sex steroids in the prescribed ratios, along with natural progesterone, is a safer regimen than conjugated equine estrogens with a synthetic progestin.

The above example is not intended to represent the sole formulation within the claims of the invention. Someone skilled in the art could propose alternative formulations within the patent claims that could achieve the same clinical benefits.

What is claimed is:

1. A method of hormone replacement therapy utilizing bio-identical sex steroids, said method comprising administration of micronized sex estrogenic steroids for 21-30 days out of each month, and the administration of placebo or no therapy for 0-7 days, whereby pathophysiologic aberrations, constitutional disturbances and impairments of quality of life parameters of menopause are ameliorated
said therapy comprising a daily dose of two estrogenic steroids, estradiol (E₂) and estriol (E₃). bio-identical to native estrogens,
said daily dose resulting in patient blood concentrations ranging from 20 to 100 pg/ml of estradiol and from, 0.5 to 5.0 ng/ml of estriol
said daily dose consisting of a higher amount by weight of estradiol (E₂) than estriol (E₃)

2. The method in claim 1, wherein estradiol comprises bio-identical natural estradiol

3. The method in claim 1, wherein estradiol comprises bio-identical natural estriol

4. The method in claim 1, wherein said therapy includes combined administration with bio-identical natural testosterone
said combined administration of testosterone resulting in a patient blood concentrations from 10 to 80 ng/dl of testosterone

5. The method of claim 1, wherein said administration is begun at or around the time of menopause.

6. The method of claim 1, wherein said administration is given by the transdermal, oral, or genital route.

7. A pharmaceutically acceptable topical or oral formulation comprising a daily dosage unit ranging from 0.25 to 7.5 mg of estradiol and 0.25 to 7.5 mg of estriol, whereby pathophysiologic aberrations, constitutional disturbances and impairments of quality of life parameters of menopause are ameliorated
said daily dose comprising a ratio by weight of estradiol to estriol ranging from 1.1:1.0 to 10.0:1.0

8. The formulation in claim 7, wherein estradiol comprises bio-identical natural estradiol

9. The formulation in claim 7, wherein said estriol comprises bio-identical natural estriol.

10. The formulation in claim 7, wherein said formulation includes combined administration of bio-identical natural testosterone
said combined formulation with testosterone comprising a daily dosage unit ranging from 0.25 to 7.5 mg estradiol, 0.25 to 7.5 mg estriol, and 0.0 to 5.0 mg testosterone
said combined formulation with testosterone comprising a ratio by weight of total estrogen to testosterone ranging from 11.0:1.0 to 0.9:1.0
